(19) United States
(12) Patent Application Publication
(10) Pub. No.: US 2009/0012359 A1
(43) Pub. Date: Jan. 8, 2009

(54) Medical instrument insertion apparatus and medical instrument insertion apparatus system

(76) Inventors: Shinsuke Tanaka, Tokyo (JP); Hironobu Takizawa, Tokyo (JP)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/665,091

(22) PCT Filed: Mar. 24, 2006
(86) PCT No.: PCT/JP06/06009

§ 371 Date: Apr. 10, 2007

(30) Foreign Application Priority Data
Mar. 28, 2005 (JP) 2005-093190
Mar. 3, 2006 (JP) 2006-057375

(51) Int. Cl. A61B 1/00

(52) U.S. Cl. 600/114

(57) ABSTRACT

An object of the present invention is to provide a medical instrument insertion apparatus and a medical instrument insertion apparatus system in which an insertion unit can securely be propelled forward in a body cavity while stabilized by a simple mechanism. The medical instrument insertion apparatus includes a helical structure unit provided in a long and thin insertion unit (10), a retaining unit (30) which retains the insertion unit (10) along a predetermined axis direction while the retaining unit (30) can proceed and withdraw, and a rotation drive unit (20) which rotates the retaining unit (30) about the predetermined axis. Therefore, operability is improved in inserting the insertion unit (10), and the insertion unit (10) can securely be inserted into the body cavity while an operator does not require complicated operation or skill.

### TECHNICAL FIELD

The present invention relates to a medical instrument insertion apparatus and a medical instrument insertion apparatus system which insert a medical instrument such as an endoscope into a curved body cavity such as a large intestine.

### BACKGROUND ART

Conventionally, in order to insert an endoscope into a deep portion of a body cavity duct such as the large intestine, generally it is necessary to allow the endoscope to pass through a complicated curved portion such as a sigmoid colon. Therefore, an operator who uses the endoscope requires skill. There are disclosed various inventions which facilitate the insertion of the endoscope.

For example, Patent Document 1 which is a first conventional example discloses a large intestine fiber scope, wherein a whole insertion unit of an endoscope is formed in a helical shape and the insertion unit is rotated by a handle provided in a side end portion of the insertion unit located outside the body, which improves the insertion property of the endoscope into the large intestine. Patent Document 2 which is a second conventional example discloses a large intestine fiber scope guide wherein many cylinders and rings are coupled to one another and a helical shaped member is provided outside the coupled cylinders and rings. In this case, the insertion unit of the endoscope is inserted into the cylinders and rings, and the coupled body including the cylinders and rings is rotated to facilitate the insertion of the endoscope into the large intestine. Patent Document 3 which is a third conventional example discloses an endoscope insertion apparatus which performs a proceeding and withdrawal operation and a twisting operation to the insertion unit of the endoscope. In this case, of plural balls pressing the endoscope insertion unit, a ball connected to a motor is rotated in an axis direction of the insertion unit or in a direction perpendicular to the axis direction, and thereby to perform the proceeding and withdrawal operation and the twisting operation in the insertion unit.

Patent Document 1: Japanese Patent Application Laid-Open No. S54-78884

Patent Document 2: Japanese Utility Model Application Laid-Open No. S51-73884

Patent Document 3: Japanese Patent Application Laid-Open No. H3-92126

### DISCLOSURE OF INVENTION

### Problem to be Solved by the Invention

However, since in the first and second conventional examples, a torque is transmitted at a rear end portion of the endoscope, the insertion unit generates unstable movement or twists between an anus which is an insertion opening and the endoscope rear end portion where a rotation transmission unit is provided. Therefore, it is difficult to improve operability. When the rotation drive unit is brought close to the insertion opening in order to improve stability of the insertion unit, a total length of the insertion unit is shortened. For this reason, it is difficult to insert the insertion unit into the deep portion of the body cavity duct. In the third conventional example, the force for impelling the endoscope is not outputted while not exceeding a predetermined value. Therefore, for example, in the case where the insertion unit of the endoscope is inserted into the deep portion of the body cavity duct, there is a problem that the impelling force runs short when a friction force generated between the insertion unit and an inner wall of the body cavity duct is increased. In order to compensate the shortage of the impelling force of the insertion unit, the apparatus is enlarged as a whole when an actuator such as a motor for generating the impelling force is made robust, which results in cost increase and poor operability. Furthermore, a drive mechanism tends to become complicated because the proceeding and withdrawal operation and the twisting operation of the insertion unit are performed by different motors.

The present invention has been made in consideration of the above-described problems, and an object of the invention is to provide a medical instrument insertion apparatus and a medical instrument insertion apparatus system in which an insertion unit can securely be propelled forward in a body cavity while stabilized by a simple mechanism.

### Means for Solving Problem

A medical instrument insertion apparatus according to one aspect of the present invention includes a helical structure unit which is provided in a long and thin insertion unit; a retaining unit which retains the insertion unit along a direction of a predetermined axis while the insertion unit can proceed and withdraw; and a rotation drive unit which rotates the retaining unit.

According to the configuration, in the state in which the retaining unit is maintained at a constant position with respect to the subject, the retaining unit retains the insertion unit in the axis direction while the insertion unit can proceed and withdraw. Because the insertion unit is retained by the retaining unit, the insertion unit is driven by following the rotation of the retaining unit. Therefore, in the body cavity duct, the helical structure unit is rotated by coming into contact with an inner wall of the duct, which allows the insertion unit to be smoothly moved.

In the medical instrument insertion apparatus according to the invention, desirably the retaining unit has a resistance portion, the resistance portion is provided at a position where the resistance portion comes into contact with the insertion unit, and the resistance portion is movable along the predetermined axis. With the configuration, the resistance portion is rotated in association with the rotation of the retaining unit. At this point, because the resistance portion is in contact with the insertion unit, the insertion unit is also rotated in association with the rotation of the resistance portion. As a result, in the case where a part of the insertion unit exists in the body cavity, an impelling force is generated in this part of the insertion unit. Because the resistance portion is moved along the predetermined axis, only the resistance portion is moved along the insertion unit even if the insertion unit is moved along the predetermined axis. Therefore, the retaining unit and the rotation drive unit are never dragged nor moved by the movement of the insertion unit. Accordingly, the retaining unit and rotation drive unit can always be maintained at constant positions.

Preferably, the resistance portion generates a resistant force in a direction substantially perpendicular to the direction of the predetermined axis. According to the configuration, the rotation of the rotation drive unit can securely be transmitted to the insertion unit by the resistance portion.

In the medical instrument insertion apparatus according to the invention, preferably the resistance portion is a belt which intermittently has protrusions in the direction along the predetermined axis. With the configuration, the proceeding and withdrawal direction of the insertion unit can be restricted.

In the medical instrument insertion apparatus according to the invention, preferably the resistance portion is a rotation member which has a rotation shaft in the direction substantially perpendicular to the predetermined axis direction. According to the configuration, the means for restricting the proceeding and withdrawal direction of the insertion unit can be provided.

In the medical instrument insertion apparatus according to the invention, the retaining unit may include a magnetic field generating unit. According to the configuration, the structure of the retaining unit can be simplified.

In the medical instrument insertion apparatus according to the invention, the medical instrument insertion apparatus has an outer diameter changing unit for changing an outer diameter of the helical structure unit. According to the configuration, the contact between the helical structure unit and the tissue surface in the body can be conducted properly, so that the insertion unit can securely be propelled forward.

A medical instrument insertion apparatus system according to another aspect of the present invention includes a long and thin insertion unit; a helical structure unit provided in the insertion unit; a retaining unit which retains the insertion unit along a direction of a predetermined axis while the insertion unit can proceed and withdraw; a rotation drive unit which rotates the retaining unit about the predetermined axis; and a medical instrument which is guided and inserted into the body cavity by the insertion unit.

According to the configuration, since the retaining unit retains the insertion unit in the axis direction while the insertion unit can proceed and withdraw, the rotation drive unit rotates the retaining unit while the rotation drive unit is maintained at a constant position with respect to the subject. At this point, when the insertion unit is retained by the retaining unit, the insertion unit is driven by following the rotation of the retaining unit. Furthermore, when the helical structure unit is provided in the insertion unit, the helical structure unit is rotated in the body cavity duct by coming into contact with the duct inner wall, which allows the insertion unit to be smoothly moved.

In the medical instrument insertion apparatus system according to the invention, preferably the retaining unit includes a resistance portion which is movable in a longitudinal direction of the insertion unit, the resistance portion resisting against the helical structure unit in a direction substantially perpendicular to the longitudinal direction of the insertion unit. According to the configuration, the rotation can securely be transmitted to a medical apparatus such as an endoscope by the resistance portion provided in the retaining unit.

In the medical instrument insertion apparatus system according to the invention, the retaining unit may include a magnetic field generating unit, and the insertion unit may include a magnet or a magnetic material. According to the configuration, the configuration of the retaining unit can be simplified.

### EFFECT OF THE INVENTION

According to the medical instrument insertion apparatus of the present invention, the rotation operation is transmitted to the insertion unit near the insertion opening, so that the insertion unit can stably be inserted into the body cavity with no unstable movement nor distortion of the insertion unit existing outside the body. As a result, insertion operability is improved, so that the insertion unit can securely be inserted into the body cavity while an operator does not require the complicated operation or skill.

According to the medical instrument insertion apparatus system of the present invention, because of the same effect described above, the insertion unit which assists the medical instrument to be inserted into the body cavity can stably be inserted into the body cavity. As a result, insertion operability is improved, so that the insertion unit can securely be inserted into the body cavity while an operator does not require the complicated operation or skill.

### BRIEF DESCRIPTION OF DRAWINGS

FIG. 1 is a view showing a state where an insertion unit of a medical instrument insertion apparatus system according to the invention is propelled forward in a body cavity duct;

FIG. 2A is a view schematically explaining an entire configuration of a medical instrument insertion apparatus system according to a first embodiment of the invention;

FIG. 2B is an enlarged view of a portion surrounded by the letter A of FIG. 2A;

FIG. 3 is a view showing details of a rotation drive unit and a retaining unit in the medical instrument insertion apparatus system of FIG. 1;

FIG. 4A is a longitudinally sectional side view showing a configuration of a caterpillar unit in the retaining unit of FIG. 3;

FIG. 4B is a longitudinally sectional front view showing the configuration of the caterpillar unit in the retaining unit of FIG. 3;

FIG. 4C is a plan view of a retaining belt;

FIGS. 5A and 5B are views showing latch deformation when the caterpillar unit is operated;

FIGS. 6A to 6D are views showing latch deformation when the caterpillar unit is operated in the opposite direction;

FIG. 7 is a view showing a state in which an endoscope is inserted into the body cavity duct using the medical instrument insertion apparatus system of FIG. 2;

FIG. 8A is a longitudinally sectional side view showing a configuration of a rotation member of a retaining unit according to a modification of the retaining unit of FIG. 3;

FIG. 8B is a longitudinally sectional front view showing a configuration of the rotation member of the retaining unit according to the modification of the retaining unit of FIG. 3;

FIG. 9A is a view showing an internal structure of a retaining unit according to a modification of the retaining unit of FIG. 3;

FIG. 9B is a longitudinally sectional side view showing a configuration of a latch according to the modification of the retaining unit of FIG. 3;

FIG. 10 is a schematic view showing a configuration of a retaining unit according to a modification of the retaining unit of FIG. 3;

FIG. 11A is a schematic view showing a configuration of a retaining unit according to a modification of the retaining unit of FIG. 3;

FIG. 11B is an enlarged sectional view showing a part of a central portion of the retaining unit according to the modification;

FIG. 12A is a schematic view showing a configuration of a retaining unit according to a modification of the retaining unit of FIG. 3;

FIG. 12B is an enlarged sectional view showing a part of the retaining unit according to the modification;

FIG. 13 is a schematic view showing an entire configuration of a medical instrument insertion apparatus system according to a second embodiment of the invention;

FIG. 14 is a view showing a configuration of an insertion unit, a hollow shaft motor, and a tubular magnet;

FIG. 15 is a view showing an internal configuration of an insertion unit according to a modification of the insertion unit of FIG. 13;

FIG. 16 is a view showing an insertion unit and a rotation drive unit according to a modification of the medical instrument insertion apparatus system of FIG. 13;

FIG. 17 is a schematic view showing an entire configuration of a medical instrument insertion apparatus system according to a third embodiment of the invention;

FIG. 18A is a view showing a transmission unit concerning the medical instrument insertion apparatus system of FIG. 17;

FIG. 18B is a sectional view showing a configuration of the transmission unit;

FIG. 19 is a view showing a state in which a forceps is inserted into the body cavity duct using the medical instrument insertion apparatus system of FIG. 17;

FIG. 20 is a schematic view showing an entire configuration of a medical instrument insertion apparatus system according to a fourth embodiment of the invention;

FIG. 21A is a sectional view showing details of a rotation transmission system and the like in the medical instrument insertion apparatus system of FIG. 20;

FIG. 21B is a sectional view taken on line A-A of FIG. 21A;

FIG. 22A is a view showing an operation of a slider in the rotation transmission system of FIG. 21;

FIG. 22B is a view showing an operation of the slider in the rotation transmission system of FIG. 21;

FIG. 22C is a view showing an operation of the slider in the rotation transmission system of FIG. 21;

FIG. 22D is a view showing an operation of the slider in the rotation transmission system of FIG. 21;

FIG. 23 is a sectional view showing a configuration of a slider according to a modification of the rotation transmission system of FIG. 21;

FIG. 24 is a sectional view showing a configuration of a rotation transmission system according to a modification of the rotation transmission system of FIG. 21;

FIG. 25 is a view showing details of a base portion and a rotation transmission system in a medical instrument insertion apparatus system according to a fifth embodiment of the invention;

FIG. 26A is a view showing an entire configuration of a medical instrument insertion apparatus system according to a modification of a helical structure unit of the invention;

FIG. 26B is an enlarged view showing a part of the insertion unit shown in FIG. 26A;

FIG. 27A is a view showing a change in shape of a helical structure unit;

FIG. 27B is a view showing the change in shape of the helical structure unit; and

FIG. 27C is a view showing the change in shape of the helical structure unit.

### EXPLANATIONS OF LETTERS OR NUMERALS

1, 100, 150, 200, 300 Medical instrument insertion apparatus system
10, 110, 160 Insertion unit
11, 112 Helical structure unit
12 Hollow tube (outer diameter changing unit)
20 Rotation drive unit
21 Motor
22 Pulley
23 Rotation transmitting belt
30 Retaining unit
31 External cylinder
33 Caterpillar unit
34 Latch
42, 243 Retaining belt
71 Endoscope
81 Rotation member
83 Load detecting unit
86 Magnet
87, 130 Tubular magnet
111 Ring magnet
120 Hollow shaft motor
121 Magnetic force generating unit
140 Capsule medical apparatus
151 High-pressure air source
152 Transmission unit
210 Base portion
220 Rotation transmission system
230i (230a, 230b) Slider
240i (240a, 240b) Belt rotation body
241i (241a, 241b) Belt rotation motor
310i (310a, 310b) Pressing member

### BEST MODE(S) FOR CARRYING OUT THE INVENTION

### First Embodiment

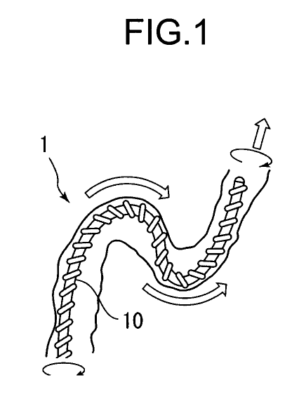

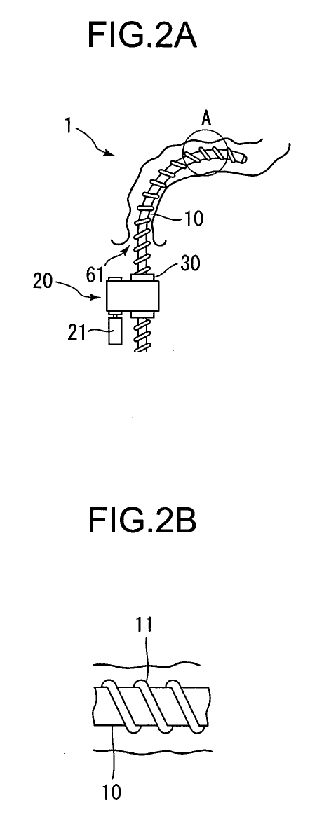

Exemplary embodiments of the present invention will be described below. A medical instrument insertion apparatus system according to a first embodiment of the invention will be described below with reference to FIGS. 1 to 12. FIG. 1 is a view showing a state in which an insertion unit of a medical instrument insertion apparatus system 1 of the invention is propelled forward in a body cavity duct. FIG. 2A is a view schematically explaining an entire configuration of the medical instrument insertion apparatus system 1, and FIG. 2B is an enlarged view of a portion surrounded by the letter A of FIG. 2A.

As shown in FIG. 2A, the medical instrument insertion apparatus system 1 includes an insertion unit 10, a rotation drive unit 20, and a retaining unit 30. The insertion unit 10 having flexibility is formed in a long and thin shape and the insertion unit 10 is inserted into the body cavity duct cavity such as the large intestine. The rotation drive unit 20 has a function of rotating the retaining unit 30. The retaining unit 30 has a function of retaining the insertion unit 10, and rotating the insertion unit 10 by being rotated upon receipt of the torque from the rotation drive unit 20.

The insertion unit 10 has the flexibility, so that the insertion unit 10 can be bent according to the shape of the body cavity duct when the insertion unit 10 is inserted into the body cavity duct. As shown in FIG. 2B, a helical structure unit 11, which has a helical shape and is formed by a string shape member, is provided in a surface of the insertion unit 10. At least a part of the helical structure unit 11 has a function of generating the impelling force, when the insertion unit 10 is rotated while the part of the helical structure unit 11 comes into contact with the inner wall of the body cavity duct. The rotation drive unit 20 includes a motor 21 for generating a torque, a pulley 22 connected to the motor 21, and a rotation transmitting belt 23 for transmitting the torque from the pulley 22 to the retaining unit 30.

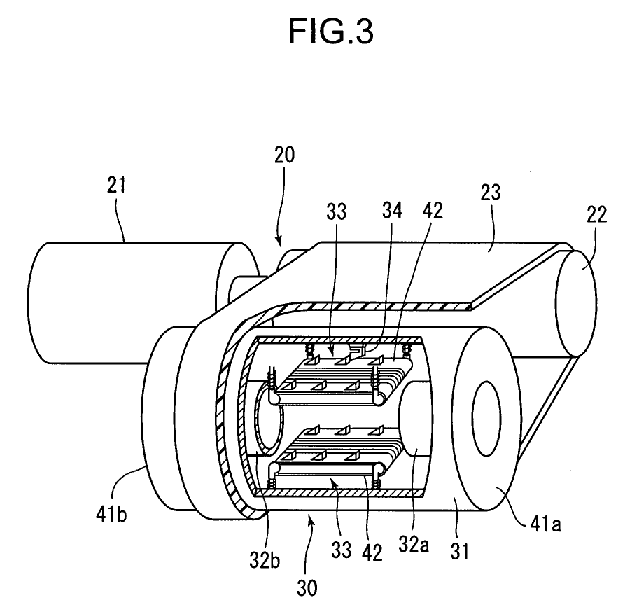

FIG. 3 is a view showing details of the rotation drive unit 20 and the retaining unit 30. The retaining unit 30 includes insertion pipes 32a and 32b, a caterpillar unit 33, and a latch 34 in an external cylinder 31 having a hollow cylindrical shape. The insertion pipes 32a and 32b have a hollow cylindrical shape such that the insertion unit 10 is inserted thereinto, and the insertion pipes 32a and 32b are fixed to end faces 41a and 41b of the external cylinder 31 respectively so as to be coaxial with a center axis (predetermined axis) of the external cylinder 31. In the external cylinder 31, the plural caterpillar units 33 are provided between the insertion pipes 32a and 32b, and arranged so as to face each other across the center axis of the external cylinder 31. The invention is not limited to the first embodiment. For example, at least three caterpillar units 33 are provided, and the caterpillar units 33 may be arranged so as to surround the center axis of the external cylinder 31. The plural latches 34 are attached to the inner wall of the external cylinder 31 so as to face the caterpillar unit 33.

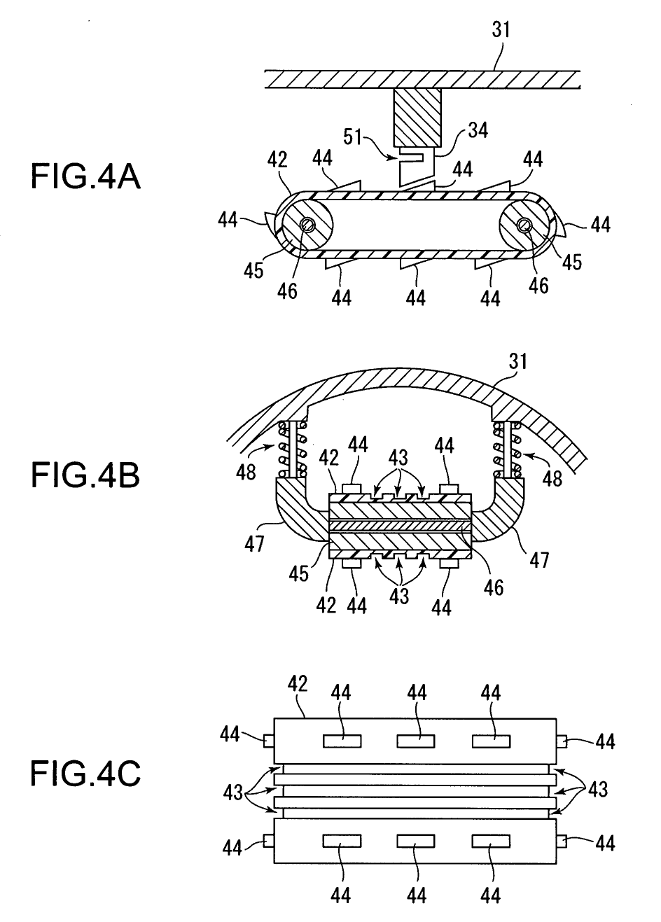

FIGS. 4A to 4C are views each showing the detail of one of plural caterpillar units 33 provided in the retaining unit 30. The caterpillar unit 33 includes a retaining belt (resistance portion) 42, a recess portion 43, a projection (convex portion) 44, and rotation cylinders 45. The retaining belt 42 serving as the resistance portion has the flexibility, and is a ring member having a width larger than an outer diameter of the insertion unit 10. The retaining belt 42 is provided while tensioned by the two rotation cylinders 45. The plural recess portions 43 are provided near the center on the surface of the retaining belt 42 along a longitudinal direction of the retaining belt 42. The projection 44 serving as the convex portion has a sawtooth shape. The plural projections 44 are arranged near both ends of the retaining belt 42 along the longitudinal direction of the retaining belt 42. As a consequence, the recess portions 43 come into contact with the insertion unit 10 inserted into the insertion pipes 32a and 32b, and the projections 44 are not in contact with the insertion unit 10. Preferably the projection 44 is formed by an elastic member because the shape of the retaining belt 42 is bent.

The rotation cylinders 45 have the cylindrical shape, and each of the rotation cylinders 45 has a length equal to or more than a width of the retaining belt 42. The rotation cylinders 45 are retained by the shaft members 46 while being rotatable about the shaft members 46, respectively. Bearings 47 are provided at both ends of the shaft member 46, and the bearings 47 are attached to the inner wall of the external cylinder 31 through springs 48. With this configuration, the caterpillar unit 33 is biased toward the center axis direction of the external cylinder 31 by an elastic force of the spring 48. That is, the insertion unit 10 to be inserted into the insertion pipes 32a and 32b is clamped with proper pressure by the plural caterpillar units 33.

Thus, the retaining belt 42 is retained while being rotatable in the longitudinal direction, so that the retaining belt 42 does not interrupt the proceeding and withdrawal of the insertion unit 10 even if the plural caterpillar units 33 bias the insertion unit 10. By the recess portions 43 provided in the retaining belt 42, the caterpillar unit 33 has a function of not resisting the insertion unit 10 in the proceeding and withdrawal direction to freely propel the insertion unit 10 but resisting the insertion unit 10 only in the circumferential direction. That is, the retaining unit 30 has a function of continuously transmitting the rotation power to the insertion unit 10 without interrupting the operation in the proceeding and withdrawal direction which always located near the insertion opening of the body cavity duct.

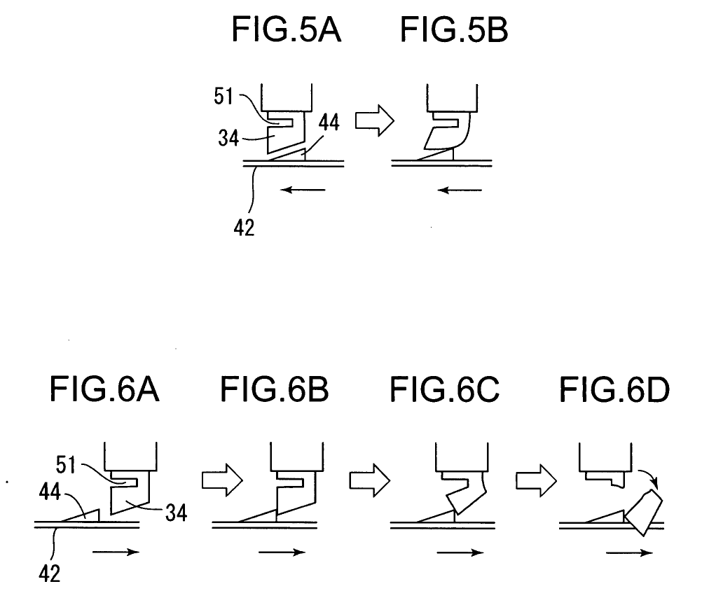

FIGS. 5 and 6 are views showing the operations of the projection 44 and latch 34. The plural latches 34 are attached to the inner wall of the external cylinder 31, and the latches 34 are arranged while facing the caterpillar unit 33 so as to come into contact with the projection 44 on the surface of the caterpillar unit 33. A notch 51 is provided in a part of the side face of the latch 34. When the caterpillar unit 33 is rotated in the direction as shown in FIGS. 5A and 5B (leftward direction in the drawings), the projection 44 is moved in association with the rotation of the caterpillar unit 33 to come into contact with the latch 34. At this point, the latch 34 is bent by the notch 51 provided in the latch 34, and the projection 44 passes through the latch 34. On the other hand, when the caterpillar unit 33 is rotated in the direction as shown in FIG. 6A (rightward direction in the drawing) to bring the projection 44 into contact with the latch 34, the caterpillar unit 33 is not rotated because the projection 44 and the latch 34 interfere with each other as shown in FIG. 6B. When in this state, the force for rotating the caterpillar unit 33 is further applied, the latch 34 is bent such that the notch 51 is split off as shown in FIG. 6C. Then, when the force is continuously applied, the latch 34 is folded down as shown in FIG. 6D. At this point, the caterpillar unit 33 can freely be rotated in both directions. Thus, while the caterpillar unit 33 is rotatable in the proceeding direction of the insertion unit 10, the caterpillar unit 33 is not rotated unless a reaction force not lower than a predetermined value is applied.

Action of the medical instrument insertion apparatus system 1 having the above configuration will be described below. Although the case where the medical instrument insertion apparatus system 1 is applied to the insertion into the large intestine is described by way of example, the medical instrument insertion apparatus system 1 can be also applied to the insertion into other body cavity ducts, and the action is similar to that described below.

As shown in FIG. 2, in the case where a medical treatment is required in a body cavity duct of a subject such as the large intestine, an operator places the rotation drive unit 20 having the retaining unit 30 incorporated therein near an anus 61 which is an insertion opening of the subject, and the operator inserts the insertion unit 10 through the insertion pipes 32a and 32b in the retaining unit 30. At this point, the rotation drive unit 20 is placed such that the side of the end face 41a of the external cylinder 31 is orientated toward the subject. Then, the operator inserts a neighbor of a front edge portion of the insertion unit 10 into the large intestine to drive the motor 21. The rotation driving force generated by the motor 21 is transmitted to the retaining unit 30 through the pulley 22 and the rotation transmitting belt 23.

In the retaining unit 30, the plural retaining belts 42 bias the insertion unit 10 inserted through the insertion pipes 32a and 32b. At this point, the insertion unit 10 is in contact with the plural recess portions 43 provided in the longitudinal direction of the retaining belt 42. Therefore, when the retaining unit 30 is rotated, the force for resisting against the insertion unit 10 is generated in the direction substantially perpendicular to the longitudinal direction of the insertion unit 10. As a result, the insertion unit 10 is rotated by following the rotation operation of the retaining unit 30, which securely transmits the rotation driving force generated by the motor 21 to the insertion unit 10.

When the insertion unit 10 starts the rotation operation, the helical structure unit 11 provided in the surface of the insertion unit 10 is rotated to generate the impelling force while at least a part of the helical structure unit 11 comes into contact with an intestine wall, so that the insertion unit 10 is smoothly propelled forward in the large intestine. In the retaining unit 30, the retaining belt 42 is moved along the predetermined axis by following the movement of the insertion unit 10, in association with the propulsion of the insertion unit 10. Therefore, the retaining unit 30 and the rotation drive unit 20 are never dragged nor moved by the movement of the insertion unit 10. That is, the retaining unit and the rotation drive unit can always be maintained at the constant positions, allowing the rotation drive unit 20 to be always placed near the anus irrespective of the total length of the insertion unit 10. As a result, because a distance between the rotation drive unit 20 and the anus 61 can be shortened, there is no risk of the unstable movement or distortion in the insertion unit 10 located between the rotation drive unit 20 and the anus 61.

When the inserted length of the insertion unit 10 is short since the insertion unit 10 starts the insertion into the body cavity duct, the impelling force generated by the helical structure unit 11 is small. For this reason, sometimes the propulsion is stopped such that the front edge of the insertion unit 10 is resisted against the intestine wall, and backing power for pulling out the insertion unit 10 to the outside of the body is applied to the insertion unit 10. In such cases, the rotation is regulated such that the withdrawal of the insertion unit 10 is prevented by the functions of the projection 44 and latch 34 which are provided in the caterpillar unit 33. Then, the insertion unit 10 is propelled forward to the deep portion of the large intestine, and a sufficient impelling force is obtained. Therefore, the power for pulling out the insertion unit 10 to the outside of the body is prevented until the insertion unit 10 obtains the sufficient impelling force, so that the insertion unit 10 can easily be inserted. On the other hand, in some cases, the insertion unit 10 is bent along the shape of the intestine, which increased the reaction force. In such cases, the latch 34 is broken and the rotation is not regulated. Consequently, the forced insertion of the insertion unit 10 is prevented.

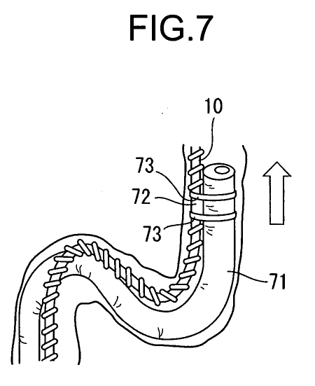

After the insertion unit 10 reaches the deepest portion of the large intestine, the operator stops the motor 21 to stop the propulsion of the insertion unit 10. Then, as shown in FIG. 7, an endoscope 71 which is the medical instrument is inserted into the large intestine for the purpose of observation, diagnosis, or treatment. A cylindrical member 72, which inserts the insertion unit 10 through the cylindrical member 72 to connect the endoscope 71 with the insertion unit 10, is provided near the front edge of the endoscope 71 by a fixing member 73. When the endoscope 71 is inserted into the body cavity duct, the end portion of the insertion unit 10 existing outside the body is inserted through the cylindrical member 72. Then, the endoscope 71 is guided to the deep portion of the large intestine along the insertion unit 10. That is, the inserted insertion unit 10 has a function as guide wire of the endoscope 71. Thus, because the insertion unit 10 is utilized as the guide wire, the endoscope 71 can smoothly be inserted to the deepest portion of the large intestine. When the endoscope 71 reaches the deepest portion of the large intestine, the insertion unit 10 may be pulled so as not to interrupt the diagnosis or treatment.

As described above, according to the medical instrument insertion apparatus system 1 of the first embodiment, the rotation drive unit 20 can rotate and drive the insertion unit 10 while maintaining the constant distance with respect to the subject. That is, the rotation drive unit 20 can always be arranged near the insertion opening of the subject irrespective of the total length of the insertion unit 10. Therefore, because the distance between the rotation drive unit 20 and the insertion opening can be shortened, the unstable movement or distortion is not generated in the insertion unit 10 located between the rotation drive unit 20 and the insertion opening. Accordingly, the insertion unit 10 can stably be inserted into the body cavity duct. That is, the insertion unit 10 can smoothly be inserted into and securely be propelled forward in the body cavity duct, when the helical structure unit 11 is propelled forward in the body cavity duct by coming into contact with the inner wall of the body cavity duct while rotated. As a result, the operator does not require the complicated operation or the skill, and the operator can securely insert the insertion unit 10.

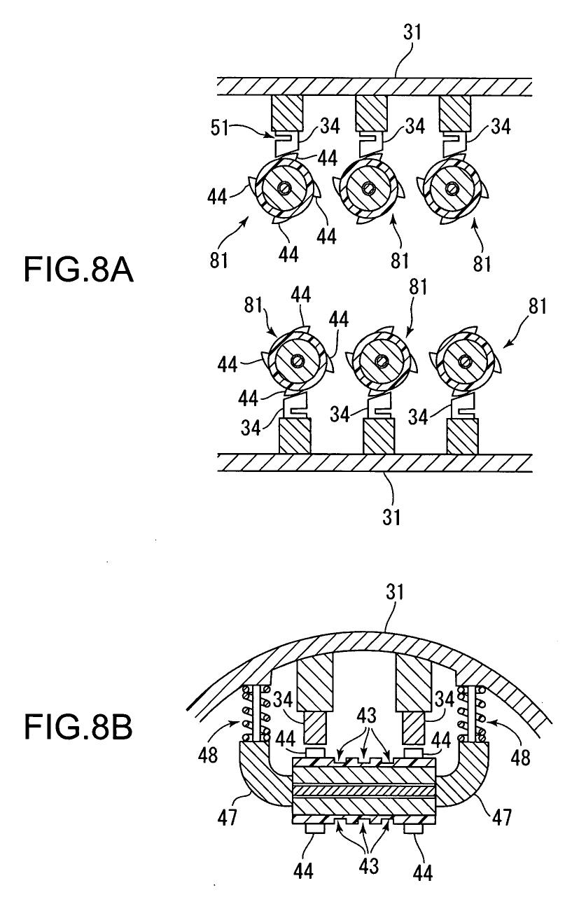

The first embodiment is not limited to the above-described configuration. First, the first embodiment is configured such that the retaining unit 30 has the caterpillar unit 33. Alternatively, as shown in FIGS. 8A and 8B, the retaining unit 30 may configured to have plural rotation member (resistance portion) 81. In this case, the rotation member 81 serving as the resistance portion is formed to be rotatable in the longitudinal direction of the insertion unit 10, and the plural rotation members 81 are arranged along the longitudinal direction. Plural recess portions 43 are provided in the surface of the rotation member 81 along the circumferential direction of the rotation member 81. As with the retaining belt 42, the width of the rotation member 81 is larger than the outer diameter of the insertion unit 10, and plural projections 44 are provided near both ends of the rotation member 81. The latch 34 is fixed to the inside of the external cylinder 31 so as to face the projection 44.

In this manner, as with the caterpillar unit 33 in the first embodiment, the rotation power can be transmitted to the insertion unit 10 without interrupting the propulsion and withdrawal of the insertion unit 10. In the body cavity duct, the helical structure unit 11 is rotated by the rotation power transmitted to the insertion unit 10 while coming into contact with the intestine wall, which allows the insertion unit 10 to proceed or withdraw. The projection 44 and the latch 34 can be attached like the caterpillar unit 33 by forming the width of the rotation member 81 larger than the outer diameter of the insertion unit 10, and the same effect as the first embodiment can be obtained.

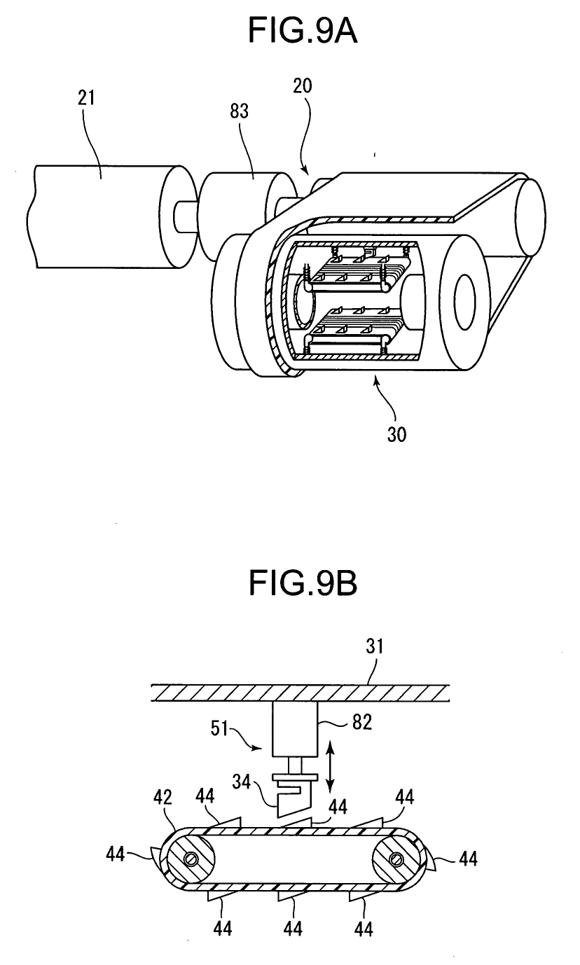

Second, the regulation in the rotation direction is released by the breakage of the latch 34 in the first embodiment. Alternatively, as shown in FIG. 9A, a load applied to the motor 21 may be detected to move the latch 34. In this case, a load detecting unit 83 which detects the load applied to the motor 21 is provided between the motor 21 and the pulley 22, and an actuator 82, which moves the latch 34 in the direction in which the latch 34 is brought close to or separated from the projection 44, is provided between the external cylinder 31 and the latch 34 as shown in FIG. 9B. When the insertion unit 10 proceeds to the deep portion of the large intestine to cause the load applied to the motor 21 to exceed a threshold, the load detecting unit 83 judges that the insertion unit 10 is inserted to the deep portion of the large intestine where the regulation in rotation direction is not required, and the load detecting unit 83 starts the drive of the actuator 82. The actuator 82 separates the latch 34 from the position where the latch 34 is in contact with the projection 44, and releases the regulation in rotation direction. Therefore, because the latch 34 is not broken in releasing the regulation in rotation direction, the retaining unit 30 can repeatedly be used.

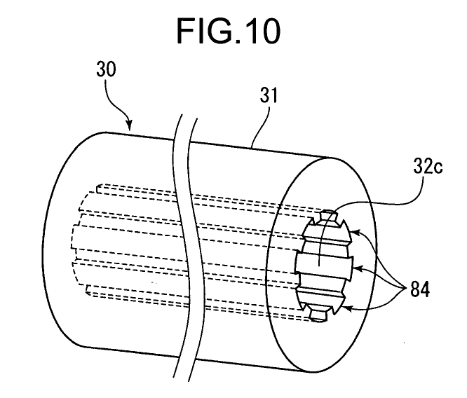

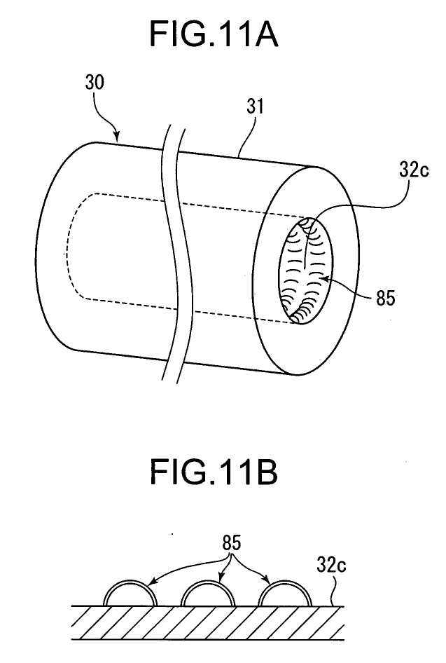

Third, in the first embodiment, the latch 34 and the projection 44 are provided as means for regulating the rotation direction. Alternatively, the means for regulating the rotation direction may be neglected. In this case, as shown in FIG. 10, the retaining unit 30 has one insertion pipe 32c which is coaxial with the center axis of the retaining unit 30, and an inner peripheral portion of the insertion pipe 32c has many grooves 84 along the longitudinal direction thereof. As shown in FIGS. 11A and 11B, the inner peripheral portion of the insertion pipe 32c may be covered with many cilia 85. Both ends of the cilia 85 are fixed to the inner peripheral portion of the insertion pipe 32c such that the cilia 85 are orientated toward the longitudinal direction of the insertion pipe 32c.

As a consequence, when the retaining unit 30 is rotated, the resistance is generated in the circumferential direction of the insertion unit 10 inserted through the insertion pipe 32c by the groove 84 or cilia 85 provided inside the insertion pipe 32c. By means of the resistance, the rotation operation of the retaining unit 30 is transmitted to the insertion unit 10 without interrupting the propulsion or withdrawal of the insertion unit 10. When the insertion unit 10 is rotated, the helical structure unit 11 is rotated while coming into contact with the intestine wall, so that the insertion unit 10 can be proceed and withdraw. At this point, because the groove 84 or cilia 85 formed inside the insertion pipe 32c is substantially parallel to the longitudinal direction of the insertion unit 10, the resistance is not generated in the proceeding and withdrawal direction of the insertion unit 10. Thus, the cost can be reduced by simplifying the configuration of the retaining unit 30.

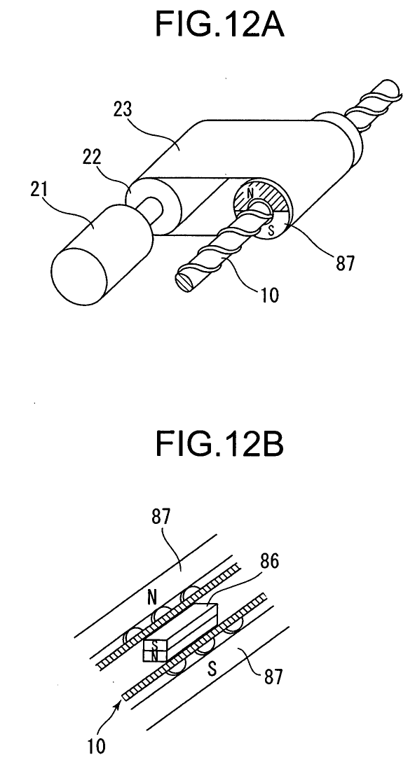

Fourth, the rotation operation of the retaining unit 30 may be transmitted to the insertion unit 10 using a magnetic force. In this case, as shown in FIGS. 12A and 12B, the insertion unit 10 has a hollow structure, and a magnet 86 is arranged in the hollow structure. The magnet 86 has a rectangular solid shape whose cross section is a square, and a diagonal line of the square has the length equal to the inner diameter of the insertion unit 10. The magnet 86 is magnetized in a radial direction of the insertion unit 10. The retaining unit 30 has a cylindrical tubular magnet 87 in place of the external cylinder 31. At this point, the magnet 86 provided in the insertion unit 10 and the tubular magnet 87 possessed by the retaining unit 30 attract each other while opposite magnetic poles face each other. Surface treatment for decreasing friction is performed to the surface of the insertion unit 10 in order that the insertion unit 10 is smoothly moved in the proceeding and withdrawal direction while inserted through the inner peripheral portion of the tubular magnet 87. With this configuration, the rotation of the motor 21 is transmitted to the tubular magnet 87, and thereby the insertion unit 10 having the magnet 86 is rotated by following the rotation of the tubular magnet 87. At this point, the helical structure unit 11 provided in the insertion unit 10 comes into contact with the inner wall of the body cavity duct while rotated, so that the insertion unit 10 is propelled forward in the body cavity duct. In this manner, the rotation drive unit 20 rotates the insertion unit 10 using the magnetic force possessed by the retaining unit 30, and consequently, the insertion unit 10 can smoothly perform the proceeding and withdrawal operation. Because the tubular magnet 87 and the magnet 86 of the insertion unit 10 attract each other, the magnet 86 is never moved with respect to the retaining unit 30.

### Second Embodiment

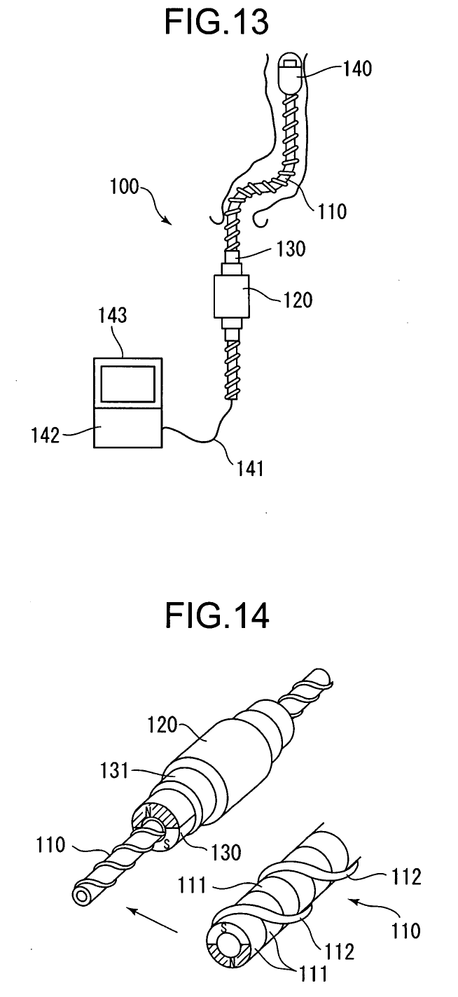

A medical instrument insertion apparatus system 100 according to a second embodiment of the invention will be described below with reference to FIGS. 13 and 14. The same components as those in the first embodiment are designated by the same numerals, and the description thereof will be omitted.

The medical instrument insertion apparatus system 100 of the second embodiment is different from that of the first embodiment in that the medical instrument insertion apparatus system 100 includes an insertion unit 110 having a hollow structure. As shown in FIG. 13, the insertion unit 110 is a tube having the flexibility, and the various functional members can be inserted through the inside of the tube. As shown in FIG. 14, the medical instrument insertion apparatus system 100 includes a hollow shaft motor 120 serving as the rotation drive unit 20 and a tubular magnet 130 serving as a retaining unit.

The insertion unit 110 is formed by coupling many thin ring magnets 111 having poles in the radial direction so as to be bendable. A helical structure unit 112 is provided in the surface of the insertion unit 110 so as to be fixed to each of the ring magnets 111. The tubular magnet 130 is a cylindrical magnet magnetized in the radial direction. In the tubular magnet 130, a cylindrical duct is provided such that the insertion unit 110 can be inserted through the inside of the duct, and the duct has a radius larger than the outer diameter of the insertion unit 110. The hollow shaft motor 120 is a cylindrical motor provided so as to be fixed to a hollow shaft 131 surrounding the tubular magnet 130, and rotates and drives the tubular magnet 130.

In assisting an capsule medical apparatus 140 as the medical instrument for observing a body cavity duct to be inserted into the body cavity duct, as shown in FIG. 13, a soft cable 141 connected to the capsule medical apparatus 140 is inserted into the tubular insertion unit 110. The capsule medical apparatus 140 has a hemispherical member whose front edge is transparent. An illumination device such as an LED for illuminating the body cavity and an imaging device such as a CCD for taking an image in the body cavity are incorporated in the capsule medical apparatus 140 while facing the hemispherical member. An electric power for driving the illumination device and imaging device is supplied through an electric power supply line provided in the cable 141. An image signal of the taken image is transmitted to an image processing device 142 installed outside the body through a signal line in the cable 141, and the processed image is displayed on a monitor 143.

Because the capsule medical apparatus 140 and the cable 141 are not fixed to the insertion unit 110, the capsule medical apparatus 140 is not rotated and the cable 141 is not distorted even if the insertion unit 110 is rotated. Thus, because only the insertion unit 110 is rotated without rotating the capsule medical apparatus 140, the capsule medical apparatus 140 is smoothly propelled forward in the body cavity duct.

Therefore, in inserting the capsule medical apparatus 140 into the body cavity duct, the operator places the hollow shaft motor 120 having the tubular magnet 130 provided therein near the anus 61 which is the insertion opening of the subject, and the operator inserts the insertion unit 110 through the inside of the tubular magnet 130. Then, the operator inserts the neighbor of the front edge of the insertion unit 110 into the large intestine to drive the hollow shaft motor 120. When the hollow shaft motor 120 is rotated, the tubular magnet 130 fixed to the inside of the hollow shaft motor 120 is rotated, and the insertion unit 110 having the ring magnet 111 is rotated by following the rotation of the magnetic field generated by the tubular magnet 130. The helical structure unit 112 provided in the surface of the insertion unit 110 is rotated while coming into contact with the inner wall of the body cavity duct by the rotation of the insertion unit 110, which generates the impelling force in the insertion unit 110. This enables the capsule medical apparatus 140 to be pushed into the deep portion of the body cavity duct. At this point, because the capsule medical apparatus 140 does not perform the rotation operation, the taken image is not rotated when the body cavity is observed with the imaging device.

As described above, according to the medical instrument insertion apparatus system 100 of the second embodiment, the insertion unit 110 can be inserted into the body cavity duct while the medical apparatus such as the capsule medical apparatus 140 is inserted through the insertion unit 110. Due to the same reason as the first embodiment, the insertion unit 110 is stably inserted into the body cavity duct, and the helical structure unit 112 comes into contact with the inner wall of the body cavity duct while rotated. Therefore, the insertion unit 110 can securely be propelled forward in the body cavity duct. As a result, the medical apparatus inserted through the insertion unit 110 can securely be inserted into the body cavity duct and propelled forward. The tubular magnet 130 serving as a retaining unit is directly rotated by the hollow shaft motor 120, so that the power transmission efficiency is improved. The effect of the second embodiment is similar to that of the first embodiment.

The second embodiment is not limited to the above-described configuration. First, the insertion unit 110 may be formed by not the ring magnet 111 but a magnetic material. Because the material used for the insertion unit 110 is not limited to the magnet, the material suitable to the insertion unit 110 can be selected.

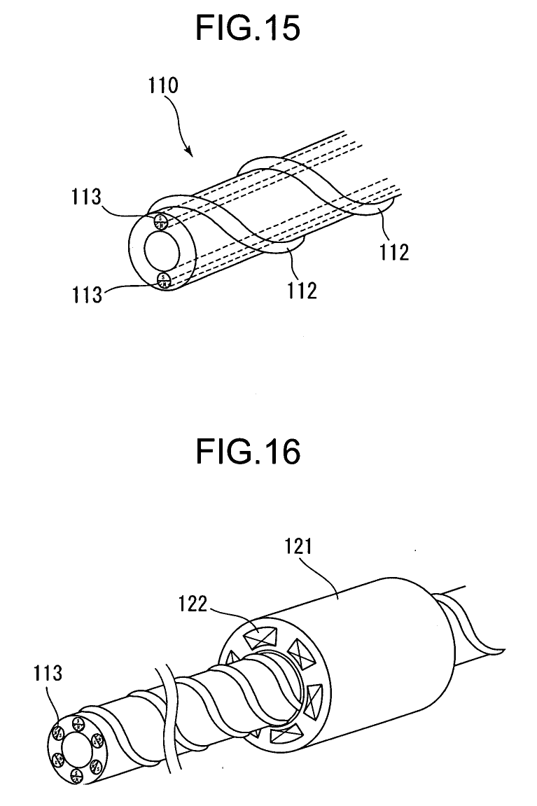

Second, the whole of the insertion unit 110 is not formed by the magnet, but a magnet having the flexibility may be provided in the insertion unit 110. For example, as shown in FIG. 15, plural string-shape soft magnets 113 may be embedded in the circumferential direction of the tubular insertion unit 110. The magnetic poles of the plural soft magnets 113 are orientated toward the center line of the insertion unit 110, respectively. The adjacent soft magnets 113 are arranged so as to have magnetization directions opposite to each other. In this case, the same effect as the second embodiment is obtained.

Third, the plural magnets may be embedded in the insertion unit 110. That is, as shown in FIG. 16, the many string-shape soft magnets 113 are embedded in the insertion unit 110, and the rotation drive unit 20 has a magnetic force generating unit 121 in place of the hollow shaft motor 120. The magnetic force generating unit 121 is configured such that many coils 122 for generating the magnetic force in the radial direction are arranged in the circumferential direction. Currents flowing into the plural coils 122 are controlled such that the adjacent coils 122 generate the magnetic forces opposite to each other in the magnetic force generating unit 121.

With this configuration, the magnetic force orientation of each of the plural coils 122 is sequentially switched by repeating the control in which the current flowing into the coil 122 is inverted. At this point, the soft magnet 113 provided in the insertion unit 110 receives the change in the magnetic force of the coil 122, which rotates the insertion unit 110. The rotation of the insertion unit 110 causes the helical structure unit 112 provided in the surface of the insertion unit 110 to be rotated while coming into contact with the inner wall of the body cavity duct, so that the impelling force is generated in the insertion unit 110, and the capsule medical apparatus 140 is pushed out toward the deep portion direction of the body cavity duct. According to the above configuration, because the number of components which are mechanically driven is decreased, a risk of failure cased by abrasion or fatigue of each component can be decreased.

### Third Embodiment

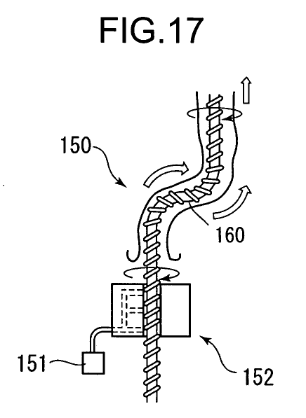

A medical instrument insertion apparatus system 150 according to a third embodiment of the invention will be described below with reference to FIGS. 17 to 19. The same components as those in the first or second embodiment are designated by the same numerals, and the description thereof will be omitted.

The medical instrument insertion apparatus system 150 of the third embodiment is different from those of the first and second embodiments in that the rotation drive unit 20 rotates the insertion unit by use of a high-pressure fluid. As shown in FIG. 17, the medical instrument insertion apparatus system 150 has a high-pressure air source 151 as a rotation drive unit and a transmission unit 152 connected to the high-pressure air source 151. The high-pressure air source 151 generates high-pressure air for rotating an insertion unit 160 to sully the high-pressure air to the transmission unit 152. The transmission unit 152 has a mechanism which blows the insertion unit 160 with the high-pressure air supplied from the high-pressure air source 151 to rotate the insertion unit 160. In the third embodiment, the insertion unit 160 is formed in the hollow structure, and the hollow structure has the inner diameter through which the endoscope 71 can be inserted.

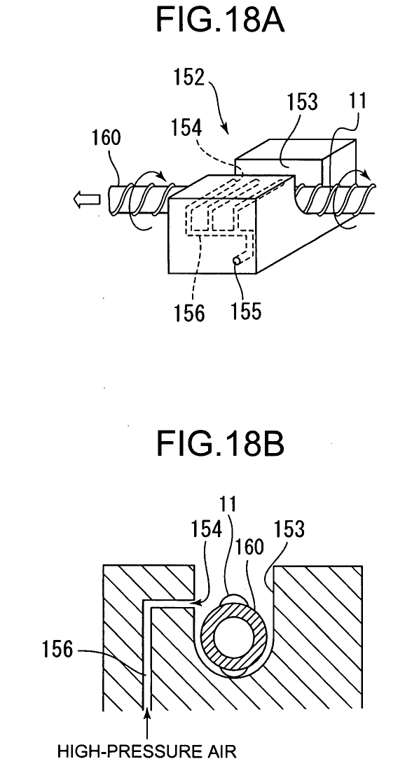

As shown in FIGS. 18A and 18B, a U-shape groove 153 serving as a retaining unit is made in the central portion of the transmission unit 152. The U-shape groove 153 has a width in which the insertion unit 160 can be slidably placed, and the U-shape groove 153 has the smooth surface such that the friction with the insertion unit 160 becomes the minimum. Plural air outlets 154 are made in the sidewall of the U-shape groove 153 at a height substantially equal to the highest position of the helical structure unit 11 provided in the surface of the insertion unit 160 when the insertion unit 160 is placed in the U-shape groove. A connection port 155 to be connected to the high-pressure air source 151 is provided in the sidewall of the transmission unit 152. The connection port 155 and the plural air outlets 154 are communicated with each other through a high-pressure duct 156a arranged in the transmission unit 152. That is, the high-pressure duct 156 connected to the connection port 155 is branched into plural ducts in the transmission unit 152, and the branched ducts are connected to the plural air outlets 154, respectively. The high-pressure duct 156 is vertically arranged near the air outlet 154 with respect to the sidewall of the U-shape groove 153, and thereby the high-pressure air is vertically blown from the sidewall of the U-shape groove 153 and the high-pressure air is blown to the helical structure unit 11 in the circumferential direction of the insertion unit 160.

Consequently, the operator places the high-pressure air source 151 and transmission unit 152 which are of the insertion opening near the anus 61, arranges the insertion unit 160 in the U-shape groove 153 provided in the transmission unit 152, and drives the high-pressure air source 151 to supply the high-pressure air to the transmission unit 152. At this point, the high-pressure air passing through the inside of the transmission unit 152 is blow to the helical structure unit 11 of the insertion unit 160 through the plural air outlets 154. The helical structure unit 11 receives the force of the high-pressure air in the circumferential direction of the insertion unit 160, which rotates the insertion unit 160. Because the helical structure unit 11 provided in the surface of the insertion unit 160 is rotated while coming into contact with the inner wall of the body cavity duct, the impelling force is generated in the insertion unit 160 to propel the insertion unit 160 forward in the body cavity duct. The U-shape groove 153 of the transmission unit 152 has low friction, so that the U-shape groove 153 does not interrupt the propulsion of the insertion unit 160, and the transmission unit 152 is not dragged in the insertion opening.

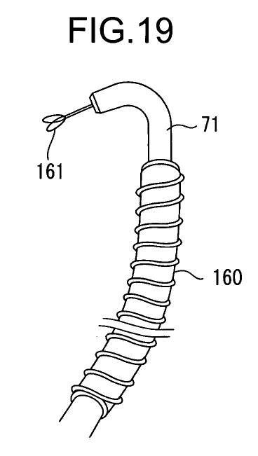

In this manner, after the insertion unit 160 having the hollow structure reaches the deep portion of the body cavity duct, the normal endoscope inspection or the endoscope treatment with forceps 161 is performed by inserting the endoscope 71 into the insertion unit 160, as shown in FIG. 19.

As described above, according to the medical instrument insertion apparatus system 150 of the third embodiment, the insertion unit 160 is rotated by using the high-pressure air. Consequently, the insertion unit 160 can be rotated by the simple structure and propelled forward in the body cavity duct. The transmission unit 152 can rotate the insertion unit 160 while the distance with the subject is always kept constant near the insertion opening of the subject.

The third embodiment is not limited to the above-described configuration. For example, the fluid with which the transmission unit 152 blows the insertion unit 160 to rotate the insertion unit 160 may be a high-pressure water flow in place of the high-pressure air. A cylindrical duct through which the insertion unit 160 can be inserted may be provided in the transmission unit 152 in place of the U-shape groove 153. In these cases, the same effect as the third embodiment is obtained.

### Fourth Embodiment

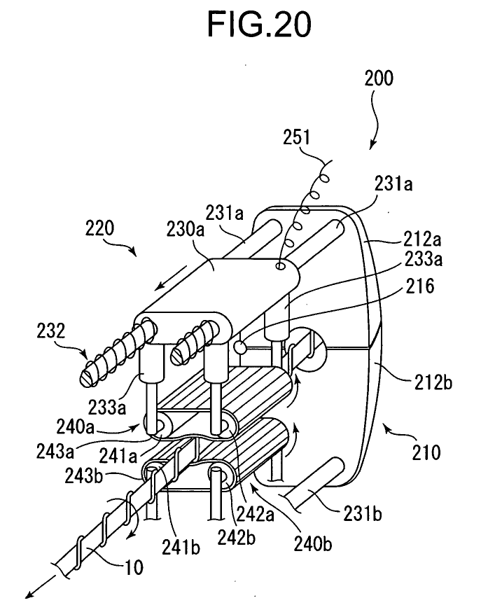

A medical instrument insertion apparatus system 200 according to a fourth embodiment of the invention will be described below with reference to FIGS. 20 to 24. The same components as those in the first embodiment are designated by the same numerals, and the description thereof will be omitted.

The fourth embodiment is different from the first embodiment in that the fourth embodiment includes a rotation control unit which actively rotates the retaining belt serving as a retaining unit to directly rotate the insertion unit 10. As shown in FIG. 20, the medical instrument insertion apparatus system 200 includes the insertion unit 10, a base portion 210 provided outside the body of the subject, and a rotation transmission system 220 connected to the base portion 210. The rotation transmission system 220 has a function of rotating the insertion unit 10.

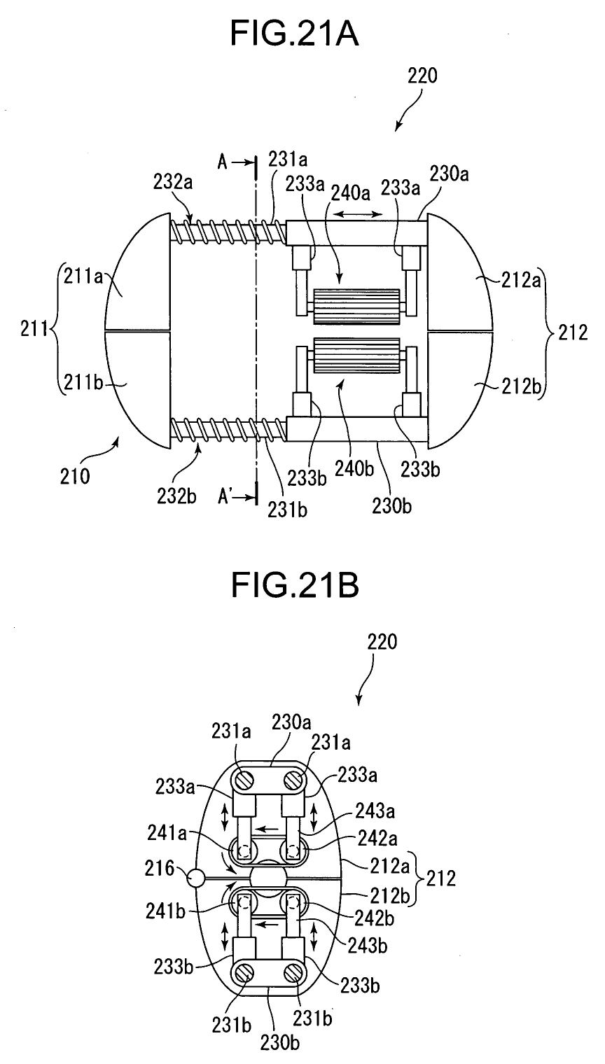

As shown in FIGS. 21A and 21B, the base portion 210 includes two pair of support members 211 and 212 which are arranged in the proceeding and withdrawal direction of the insertion unit 10. The support member 211 (212) is divided into two support members 211a (212a) and 211b (212b) having symmetric shapes. The support members 211a (212a) and 211b (212b) are connected by a hinge 216 while being openable and closable. Semi-cylindrical notches through which the insertion unit 10 is made to pass are provided near the center of the end faces on the side where the support members are brought into contact with each other when the support members are closed. That is, when the support member 211a (212a) and the support member 211b (212b) are closed, the support member 211 (212) is formed so as to make the hole having the substantially circular shape through which the insertion unit 10 is allowed to pass near the center.

In the description concerning the following embodiments, “a” is suffixed to the numeral in the configuration provided on the side of the support member 211a, “b” is suffixed to the numeral in the configuration provided on the side of the support member 211b, and “i” is suffixed to the numeral when both “a” and “b” are designates.

The rotation transmission system 220 has a slider 230i (230a and 230b) and a belt rotation body 240i (240a and 240b). The slider 230i is provided between the support member 211i and the support member 212i, and the slider 230i is moved in the proceeding and withdrawal direction of the insertion unit 10. The belt rotation body 240i is connected to the slider 230i, and the belt rotation body 240i has a function of rotating the insertion unit 10.

The slider 230i is movably provided on a slider shaft 231i provided between the support member 211i and the support member 212i. A spring 232i for biasing the slider 230i toward the direction of the support member 212i is also arranged on the slider shaft 231i between the support member 211i and the slider 230i. A linear encoder (not shown) is incorporated between the slider 230i and the slider shaft 231i. The linear encoder measures the moving distance of the slider 230i on the slider shaft 231i to detect the positional distance between the support member 211i (212i) and the slider 230i.

Plural linear actuators 233i are attached onto the side of the slider 230i opposite the insertion unit 10. In the fourth embodiment, the configuration in which the four linear actuators 233i are attached to the slider 230i is described by way of example. The linear actuator 233i drives the belt rotation body 240i in the direction in which the linear actuator 233i is brought close to and separated from the insertion unit 10 which is passed through the notch provided in the support members 211i and 212i. Thus, the slider 230i can be moved on the slider shaft 231i while integral with the belt rotation body 240i through the linear actuator 233i.

The belt rotation body 240i includes a belt rotation motor 241i constituting the rotation drive unit, a rotor 242i, and a retaining belt (retaining unit and resistance portion) 243i. The belt rotation motor 241i is connected through belt rotation shafts 244i to the two linear actuators 233i which are separated in the proceeding and withdrawal direction of the insertion unit 10 among the above-described four linear actuators 233i. The remaining two linear actuators 233i are connected to the rotor 242i through the belt rotation shaft 244i. The retaining belt 243i serving as a retaining unit and resistance portion is a ring member having the flexibility, and is tensioned by the belt rotation motor 241i and the rotor 242i. That is, the belt rotation bodies 240a and 240b are arranged so as clamp the insertion unit 10 which is passed through the notch provided in the support members 211i and 212i. The rotation speed of the belt rotation body 240i and the position on the slider shaft 231i are controlled in a synchronous manner.

Therefore, the retaining belt 243i is configured to bias the insertion unit 10 with the proper load, when the belt rotation body 240i is moved in the direction in which the belt rotation body 240i is brought close to the insertion unit 10 by the linear actuator 233i. The load biasing the insertion unit 10 is adjusted by the movement of the linear actuator 233i. The belt rotation bodies 240i are symmetrically arranged with respect to the insertion unit 10, so that the belt rotation bodies 240i can clamp the insertion unit 10 with proper pressure.

An input and output line 255 is connected to the outside through the slider 230i, and the input and output line 255 transmits the signal and power which are inputted to and outputted from the linear encoder, the belt rotation motor 241i, and the linear actuator 233i.

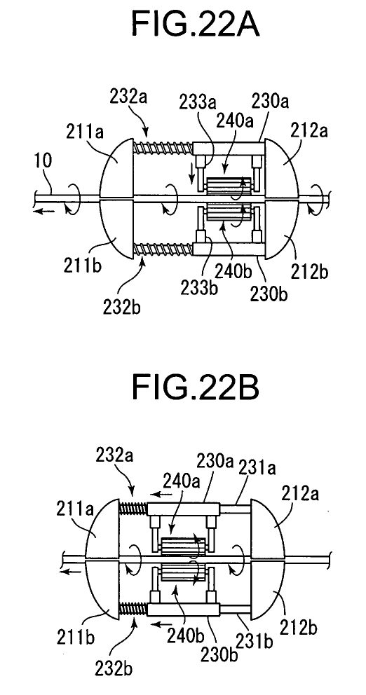

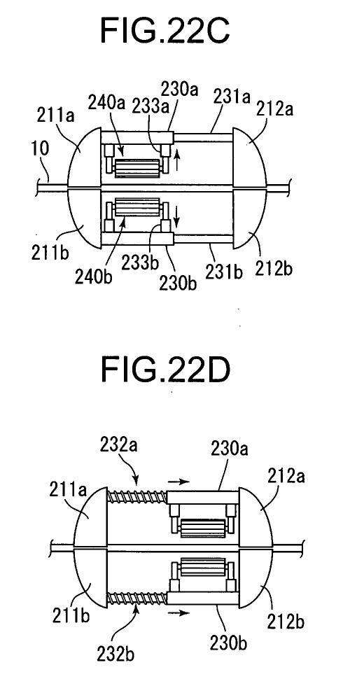

The action of the medical instrument insertion apparatus system 200 having the above-described configuration will be described below with reference to FIGS. 22A to 22D. Although the case where the medical instrument insertion apparatus system 200 is applied to the insertion into the large intestine is described by way of example, the medical instrument insertion apparatus system 200 can be also applied to the insertion into other body cavity ducts, and the action is similar to that described below. The helical structure unit 11 provided in the outer surface of the insertion unit 10 is neglected in FIGS. 22A to 22D.

First, the operator arranges the rotation transmission system 220 provided in the base portion 210 near the anus 61 which is the insertion opening such that the side of the support member 211i is orientated toward the subject. The operator opens the support member 211i about the hinge 216 to arrange the insertion auxiliary tool in the semi-cylindrical notch provided in the support member 211i, and closes the support member 211i. At this point, the support member 211i (212i) is integrally opened and closed along with other configurations such as the slider 230i and the belt rotation body 240i. Then, the operator drives the belt rotation motor 241i serving as the rotation drive unit to rotate the retaining belt 243i.

When the retaining belt 243i starts the rotation, as shown in FIG. 22A, the slider 230i is located on the side of the support member 212i of the slider shaft 231i while being integral with the belt rotation body 240i and the linear actuator 233i. Then, the belt rotation bodies 240a and 240b bias the loads to the insertion unit 10 with the linear actuators 233i so as to face each other. In this state of things, the belt rotation motor 241i is rotated and the retaining belt 243i is rotated in the direction substantially perpendicular to the proceeding and withdrawal direction of the insertion unit 10, and thereby the helical structure unit 11 (not shown) is rotated in the insertion unit 10. At this point, the rotation speed of the belt rotation body 240i and the position on the slider shaft 231i are controlled in the synchronous manner, so that the insertion unit 10 is smoothly rotated.

When the insertion unit 10 is rotated in the body cavity duct such as an alimentary canal, the helical structure unit 11 provided in the outer surface of the insertion unit 10 is rotated while coming into contact with the inner wall of the body cavity duct, which generates the impelling force in the insertion unit 10. This enables the insertion unit 10 to be propelled forward in the body cavity duct. In association with the propulsion of the insertion unit 10, the force for moving the belt rotation body 240i toward the side of the support member 211i along with the insertion unit 10 is applied to the belt rotation body 240i which transmits the rotation power to the insertion unit 10, so that the slider 230i is moved toward the side of the support member 211i while being integral with the belt rotation body 240i (FIG. 22B). At this point, the force of the spring 232i biasing the slider 230i toward the side of the support member 212i is set weaker than the impelling force of the insertion unit 10 by the helical structure unit 11. For this reason, the biasing force does not interrupt the movement of the slider 230i.

The moving distance of the slider 230i is measured by the linear encoder. When the slider 230i is moved to almost hit the support member 211i, the linear encoder detects that the slider 230i is brought close to the support member 211i. At this point, the linear actuator 233i is driven to lift the belt rotation body 240i to the height where the belt rotation body 240i is not in contact with the insertion unit 10, and the linear actuator 233i tentatively stops the transmission of the rotation power from the belt rotation body 240i to the insertion unit 10 (FIG. 22C). Therefore, the belt rotation body 240i does not follow the propulsion of the insertion unit 10, so that the slider 230i can freely be moved. Then, the slider 230i is returned to the side of the support member 212i by the biasing force of the spring 232i while being integral with the belt rotation body 240i (FIG. 22D).

When the linear encoder detects that the slider 230i hits the support member 212i, the linear actuator 233i brings the belt rotation body 240i into contact with the insertion unit 10 again, and the rotation of the retaining belt 243i by the belt rotation motor 241i is resumed to rotate the insertion unit 10. By repeating the above operations, the insertion unit 10 is smoothly continuously propelled forward in the body cavity duct.

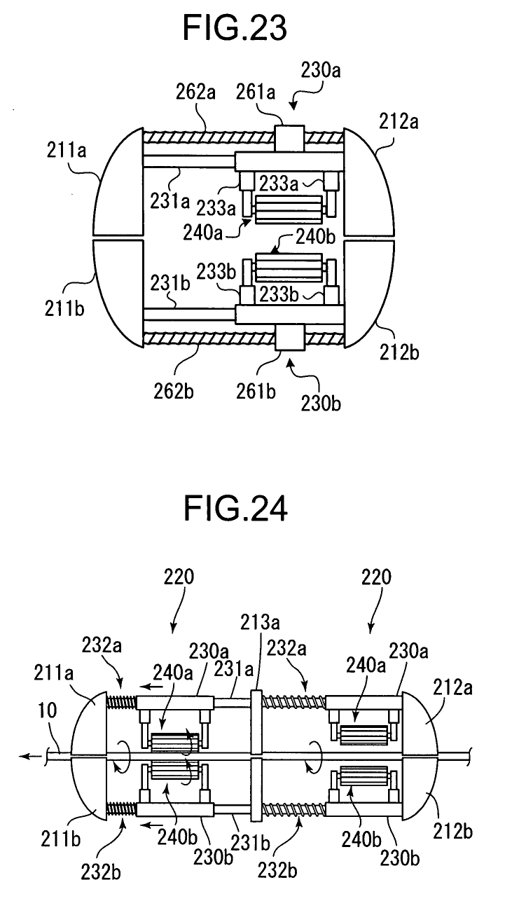

When the linear actuator 233i moves up and down the belt rotation body 240i, the retaining belt 243i may always be rotated without stopping the belt rotation motor 241i. As shown in FIG. 23, the slider 230i may be moved toward the side of the support member 212i by a linear motor 261i provided in the slider 230i in place of the spring. In this case, the drive of the linear motor 261i is stopped to freely move the slider 230i when the rotation power is transmitted to the insertion unit 10 (states shown in FIGS. 22A and 22B), and the linear motor 261i is driven to move the slider 230i only when the slider 230i is returned to the side of the support member 212i (states shown in FIGS. 22C and 22D). With this configuration for control, the same action as that of the fourth embodiment is obtained.

As described above, according to the medical instrument insertion apparatus system 200 of the fourth embodiment, the insertion unit 10 can be rotated more securely because the rotation of the insertion unit 10 is transmitted directly and actively by the retaining belt 243i. The belt rotation body 240i and the insertion unit 10 proceed integrally toward the insertion opening side by the slider 230i, and thereby the belt rotation body 240i does not interrupt the progress of the insertion unit 10 in the body cavity duct. Therefore, the insertion unit 10 can be propelled forward more securely.

The fourth embodiment is not limited to the above-described configuration. First, as shown in FIG. 24, the plural rotation transmission systems 220 may be provided along the proceeding and withdrawal direction of the insertion unit 10. That is, in the modification, an intermediate support member 213i is provided at an intermediate position between the support members 211i and 212i, and the rotation transmission systems 220 are arranged in a space between the support member 211i and the intermediate support member 213i and a space between the support member 212i and the intermediate support member 213i, respectively.

As a consequence, unlike the one set of the rotation transmission systems 220, the plural rotation transmission systems 220 can alternately be driven to rotate the insertion unit 10. When the belt rotation body 240i of one of the rotation transmission systems 220 is separated from the insertion unit 10, the other rotation transmission system 220 can transmit the rotation power to the insertion unit 10. Accordingly, because the insertion unit 10 can always be rotated, loss of a propulsion time is eliminated to efficiently propel the insertion unit 10. Other effects are similar to those of the fourth embodiment.

Second, a contact sensor (not shown) which detects the contact may be provided at one end of the slider 230i in place of the linear encoder which detects the position of the slider 230i. The contact of the slider 230i with the support member 211i is detected by the contact sensor, and the linear actuator 233i can be operated in response to the detection result. Examples of the contact sensor include a pressure sensor, an optical sensor, and a switch. However, the contact sensor is not particularly limited to the type as long as the sensor can detect that the slider 230i and the support member 211i are brought close to each other. The contact sensor is not mounted at one end of the slider 230i, but the contact sensor may be mounted at the position where the support member 211i faces the slider 230i. Therefore, the contact sensor is efficiently used because it is not necessary to always detect the position of the slider 230i unlike the linear encoder.

### Fifth Embodiment

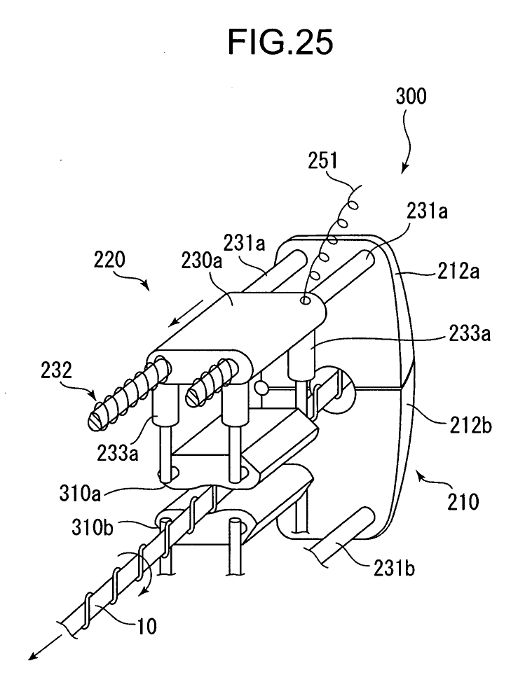

A medical instrument insertion apparatus system 300 according to a fifth embodiment of the invention will be described below with reference to FIG. 25. The same components as those in the first or fourth embodiment are designated by the same numerals, and the description thereof will be omitted.

The fifth embodiment is different from the fourth embodiment in that the base portion 210 and the rotation transmission system 220 are provided in the retaining unit 30 in the first embodiment. As with the medical instrument insertion apparatus system of the first embodiment, the medical instrument insertion apparatus system 300 includes the insertion unit 10, the rotation drive unit 20, and the retaining unit 30. The retaining unit 30 includes the base portion 210 and the rotation transmission system 220.

In this case, as shown in FIG. 25, the rotation transmission system 220 includes a pressing member 310i which does not perform the rotation operation but presses the insertion unit 10, in place of the belt rotation body 240i and the belt rotation motor 241i. The support member 211i of the base portion 210 is fixed to one end of the external cylinder 31, and the support member 212i is fixed to the other end of the external cylinder 31. Thus, the base portion 210 and the whole of the rotation transmission system 220 are fixed to the external cylinder 31 of the retaining unit 30, and thereby the base portion 210 and the whole of the rotation transmission system 220 are rotated along with the retaining unit 30.

The configuration causes the pressing members 310i to be driven by the linear actuators 233i, respectively, and to clamp the insertion unit 10 with proper pressure. The whole of the rotation transmission system 220 is rotated by the rotation drive unit 20, and thereby the rotation power is transmitted to the insertion unit 10 through the pressing member 310i. Thus, the insertion unit 10 is propelled forward in the body cavity duct while rotated. In this case, the operations of the slider 230i, the pressing member 310i, and the like in the propulsion direction of the insertion unit 10 are similar to those of the fourth embodiment.

As described above, according to the medical instrument insertion apparatus system 300 of the fifth embodiment, the rotation drive unit 20 can rotate the insertion unit 10 while the distance with the subject is kept constant near the insertion opening of the subject. The pressing member 310i and the insertion unit 10 proceed integrally toward the insertion opening side by the slider 230i, and the pressing member 310i does not interrupt the progress of the insertion unit 10 in the body cavity duct, so that the insertion unit 10 can be propelled forward more securely. Because the rotating motor 21 is arranged outside the retaining unit 30, the motor having the high output power can be used. Therefore, the insertion unit 10 can be rotated and propelled forward more securely.

The invention is not limited to the above embodiments, but various changes, modifications, partial combinations of the embodiments could be made without departing from the scope of the invention.

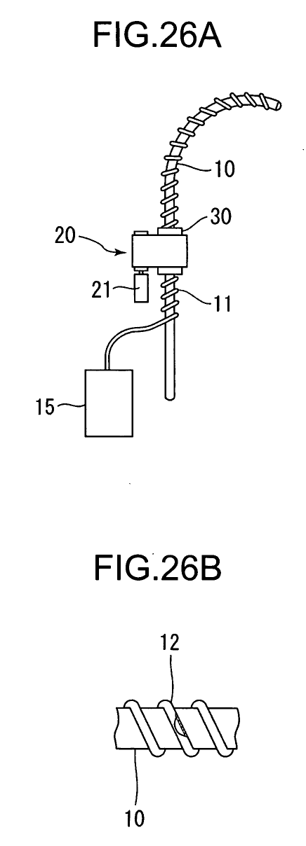

The helical structure unit 11 (112) described in each of the embodiments is not limited to the above-described modes. FIG. 26A is a view showing an entire configuration of a medical instrument insertion apparatus system according to a modification of the helical structure unit 11, and FIG. 26B is an enlarged view showing a part of the insertion unit 10 shown in FIG. 26A. In the modification, the helical structure unit 11 includes an outer diameter changing unit. That is, as shown in FIG. 26B, the helical structure unit 11 is formed by a hollow tube (outer diameter changing unit) 12 having a hollow portion and formed by an elastic member such as rubber having good stretching properties. As shown in FIG. 26A, a fluid supply unit 15 is provided at one end of the hollow tube 12 on the outside of the body. The fluid supply unit 15 has a function of supplying the fluid such as compressed air to the hollow portion formed in the hollow tube 12.

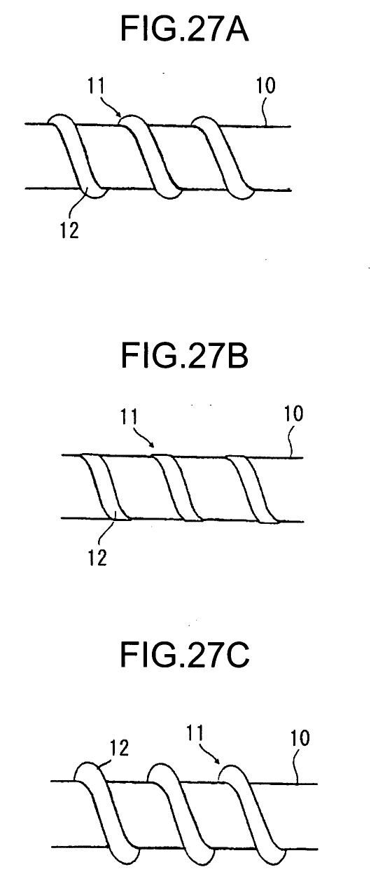

In the above configuration, when the fluid supply unit 15 is driven to supply the compressed air into the hollow tube 12, the hollow tube 12 having the good stretching properties forms a helical projection projected from the outer diameter of the insertion unit 10, as shown in FIG. 27A. On the other hand, when the drive of the fluid supply unit 15 is stopped not to supply the compressed air, the hollow tube 12 is shrunk by the elastic force of itself, as shown in FIG. 27B. For this reason, the height of the hollow tube 12 becomes substantially equal to the surface of the insertion unit 10. As shown in FIG. 27C, the outer diameter of the hollow tube 12 is increased by increasing the amount of compressed air supplied to the hollow tube 12, the height of the helical projection is larger than that of FIG. 27A. In this manner, the height of the helical projection formed by the hollow tube 12 is adjusted by adjusting the amount of compressed air supplied to the hollow tube 12. The fluid supply unit 15 may have a function of discharging the fluid from the hollow portion of the hollow tube 12.

As described above, according to the modifications, the fluid supply and the supply stop of the compressed air to the hollow tube 12 constituting the helical structure unit 11 are controlled. As a consequence, the height of the helical projection projected from the surface of the insertion unit 10 can be adjusted while the selection whether or not the helical projection is formed can be made. Accordingly, as shown in FIG. 27A or 27C, in inserting the insertion unit 10 into the body cavity duct, the hollow tube 12 can form the helical projection to improve the impelling force of the insertion unit 10 in the body cavity. In pulling the insertion unit 10 from the body cavity duct, as shown in FIG. 27B, the insertion unit 10 can smoothly be pulled smoothly in a short time by flattening the surface of the insertion unit 10.

### INDUSTRIAL APPLICABILITY

The present invention is useful to a medical instrument insertion apparatus and a medical instrument insertion apparatus system in which a medical instrument is inserted into a curved body cavity such as large intestine. Particularly, the invention is suitable to the insertion of an endoscope or an capsule medical apparatus.

1. A medical instrument insertion apparatus, comprising:
a helical structure unit which is provided in a long and thin insertion unit;
a retaining unit which retains the insertion unit along a direction of a predetermined axis while the insertion unit can proceed and withdraw; and
a rotation drive unit which rotates the retaining unit.

2. The medical instrument insertion apparatus according to claim 1, wherein the retaining unit has a resistance portion, the resistance portion is provided at a position where the resistance portion comes into contact with the insertion unit, and the resistance portion is movable along the predetermined axis.

3. The medical instrument insertion apparatus according to claim 2, wherein the resistance portion generates a resistant force in a direction substantially perpendicular to the direction of the predetermined axis.

4. The medical instrument insertion apparatus according to claim 2, wherein the resistance portion is a belt which intermittently has protrusions in a direction along the predetermined axis.

5. The medical instrument insertion apparatus according to claim 2, wherein the resistance portion is a rotation member which has a rotation shaft in a direction substantially perpendicular to the direction of the predetermined axis.

6. The medical instrument insertion apparatus according to claim 1, wherein the retaining unit includes a magnetic field generating unit.

7. The medical instrument insertion apparatus according to claim 1, wherein the medical instrument insertion apparatus has an outer diameter changing unit which changes an outer diameter of the helical structure unit.

8. The medical instrument insertion apparatus system, comprising:
a long and thin insertion unit which is inserted into a body cavity;
a helical structure unit which is provided in an outer periphery of the insertion unit;
a retaining unit which retains the insertion unit along a predetermined axis direction while the insertion unit can proceed and withdraw;
a rotation drive unit which rotates the retaining unit; and
a medical instrument which is guided and inserted into the body cavity by the insertion unit.

9. The medical instrument insertion apparatus system according to claim 8, wherein the retaining unit includes a resistance portion which is movable in a longitudinal direction of the insertion unit, the resistance portion resisting against the helical structure unit in a direction substantially perpendicular to the longitudinal direction of the insertion unit.

10. The medical instrument insertion apparatus system according to claim 8, wherein the retaining unit includes a magnetic field generating unit, and the insertion unit includes a magnet.

11. The medical instrument insertion apparatus system according to claim 8, wherein the retaining unit includes a magnetic field generating unit, and the insertion unit includes a magnetic material.
